(19) United States
(12) Patent Application Publication
(10) Pub. No.: US 2006/0249161 A1
(43) Pub. Date: Nov. 9, 2006

(54) Methods and apparatus for nasal aspiration

(76) Inventors: Kurt Waters, Brainerd, MN (US); Kent Feltz, Baxter, MN (US)

Correspondence Address: SMITH LAW OFFICE, 440 SCIENCE DR., SUITE 302, MADISON, WI, 53711, US

(21) Appl. No.: 11/298,918

(22) Filed: Dec. 9, 2005

Related U.S. Application Data
(60) Provisional application No. 60/634,702, filed on Dec. 9, 2004.

(51) Int. Cl. A61M 15/08; A61M 16/00

(52) U.S. Cl. 128/207.18; 128/200.26

(57) ABSTRACT

Apparatus and methods for withdrawing nasopharyngeal fluid samples from a patient are disclosed. A kit including a catheter and a manual suction device is disclosed, wherein the catheter can be self-navigating and define aspiration holes through which aspiration fluid can be emitted and nasopharyngeal fluid samples can be withdrawn. The catheter may also include an insertion depth scale to ensure patient comfort and optimum sample quality. The methods for obtaining nasopharyngeal fluid samples include the steps of inserting the catheter into a patient's nasopharynx, and aspirating the nasal pharynx to withdraw a specimen.

This application claims the benefit of Provisional Application No. 60/634,702 filed Dec. 9, 2004, the disclosure of which is incorporated by reference herein.

This invention relates generally to methods and apparatus for gathering samples used for diagnosing viral and bacterial infections in mammals, particularly humans. More particularly the present invention relates to methods and apparatus for collecting nasopharyngeal specimens quickly and with reduced patient discomfort.

### BACKGROUND OF THE INVENTION

Diagnosing viral and bacterial pathogens such as influenza, respiratory syncytial virus (RSV), and pertussis, requires culturing or using rapid tests of specimens gathered from a patient's throat or nasal cavity.

Studies show that nasopharyngeal aspiration provides specimens that are superior to those obtained by nasal swabs, nasal washes or throat swabs. See: Schmid et al., Prospective comparative study of culture specimens and methods in diagnosing influenza in adults, BMJ 1998;316:275; Stensballe, et al., Comparison of nasopharyngeal aspirate and nasal swab specimens for detection of respiratory syncytial virus in different settings in a developing country, Tropical Medicine and International Health 2002;7:4:317-321; and Friedman, et al., Development of a Rapid Diagnostic Test for Pertussis: Direct Detection of pertussis Toxin in Respiratory Secretions, J. of Clinical Microbiology 1989;27:11:2466-2470. Nonetheless, nasopharyngeal aspiration is considered by most practioners to be overly invasive and causing considerable patient discomfort. Further obtaining such specimens is difficult for even skilled practioners because the catheters, suctions, or IV tubing used to collect the specimens are not intended for obtaining such samples.

One prior sample collection apparatus is a tracheal suction catheter. Such a device connects to a wall-mounted suction unit with an in-line sputum trap to collect a specimen. The catheter is inserted into the nasal cavity, a specimen is withdrawn using suction rinsed or aspirated with saline. The sputum trap contents form the sample to be lab tested.

Modified tracheal suction catheters can be large and traumatic upon insertion in the nasal cavity making such a device painful for the patient, particularly children. This makes tracheal catheters unreliable because they do not always reach the nasopharynx due to the discomfort experienced by the patient with insertion. This typically results in a “less than ideal” nasal specimen rather than a nasopharyngeal specimen thereby compromising the specimen results. Tracheal catheters are also complicated in preparation and expensive due to all the components required for the collection with wall suction.

Another device used to collect nasopharyngeal specimens is a modified intravenous (IV) tube. Health care personnel typically cut off the tube end; attach it to a syringe or suction; and insert into the nasal cavity to withdraw a specimen. In addition to the catheter stiffness causing discomfort, the modified IV catheter can irritate sensitive nasal linings during insertion because the leading end has been cut and has relatively sharp edges. Similar to the tracheal suction catheter, this commonly results in a nasal rather than a nasopharyngeal specimen, thereby compromising the specimen integrity.

The open leading end of the IV tube can also be sealed against the nasal lining when fully inserted occluding the catheter and prolonging or preventing the sample collection.

Therefore, there is a need for methods and apparatus for collecting nasopharyngeal specimens that are simple for practitioners to use and have less discomfort to patients.

### SUMMARY OF THE INVENTION

The present invention is directed to improve nasopharyngeal aspiration methods and apparatuses. The methods and apparatus are relatively simple for practioners to perform and have less discomfort to patients than prior art methods and apparatus for obtaining such samples. Such improved methods and apparatus result in better samples being obtained for testing, and therefore provide more reliable results that lead to better patient treatment.

A nasal aspiration kit for obtaining a nasopharyngeal sample in accordance with the present invention includes: a manually operable suction device; and a catheter for connection to the manually operable suction device for navigating to the nasopharynx. The kit can include a lubricant for application to the catheter, a specimen container, and/or instructions that further the ease with which the kit is used. The catheter may also have a depth insertion scale for accurately reaching the nasopharynx.

The present invention also includes a method for obtaining a nasopharyngeal sample from a patient, the method comprising the steps of: positioning the patient properly, inserting a catheter into the nasal cavity of a patient, injecting a saline solution into the patient's nasal cavity; and withdrawing a specimen from the patient's nasal cavity using a manually operable suction source.

### BRIEF DESCRIPTION OF THE DRAWINGS

FIG. 1 is an aspiration kit using a syringe as a manually operated suction device in accordance with present invention;

FIG. 2 is an alternate embodiment of an aspiration kit in accordance with the present invention using a bulb aspirator as a manually operated suction device;

FIG. 3 is a side view of a Luer catheter for use in the present invention;

FIG. 4 is a bulb aspirator with a friction-fit catheter in accordance with the present invention;

FIG. 5 is a partial perspective view of a catheter in accordance with the present invention;

FIG. 6 is a partial side view of the catheter tip in FIG. 5;

FIG. 7 is a cross-sectional view of a catheter taken along line 7-7 in FIG. 6;

FIG. 8 is a partial perspective view of a catheter tip in accordance with the present invention;

FIG. 9 is a side view of a catheter with a depth insertion gauge in accordance with the present invention;

FIG. 10 is a side view of a Luer catheter with a depth insertion scale in accordance with the present invention;

FIG. 11 is a side view of a catheter with a depth insertion scale and a bulb aspirator in accordance with the present invention;

FIG. 12 is a series of instructions for performing a method in accordance with the present invention; and

FIG. 13 is a partial cross-section of a nasal cavity having in it a catheter in accordance with the present invention.

### DETAILED DESCRIPTION OF THE PREFERRED EMBODIMENTS

In the following detailed description of the preferred embodiments, the same reference numerals will be used to identify the same or similar elements in each of the figures.

Referring generally to FIGS. I and 2, there is depicted a nasal aspiration kit 20 in accordance with the present invention, including a catheter 22, a manually operable suction device 24, a lubricant 26, a vial 28 (FIG. 2 only) of aspiration fluid, and a specimen container 30.

The combination of these components in the kit 20 enables health care users to obtain consistently reliable nasopharyngeal specimens from patients while causing relatively little discomfort compared to previous apparatus for obtaining nasopharyngeal specimens. The kit 20 includes all that is necessary for obtaining and storing a specimen. It is packaged in a sealed container to ensure that it is sterile before use. Given the relatively low cost of the kit 20, it is also disposable after use.

The kit 20 includes instructions that explain the simple usage of the kit 20 components so that consistent samples can be obtained even by inexperienced medical personnel.

The catheter 22 is preferably flexible enough to minimize patient discomfort, yet rigid enough to be navigated through the nose and nasal cavity to obtain a specimen from the desired nasopharyngeal location.

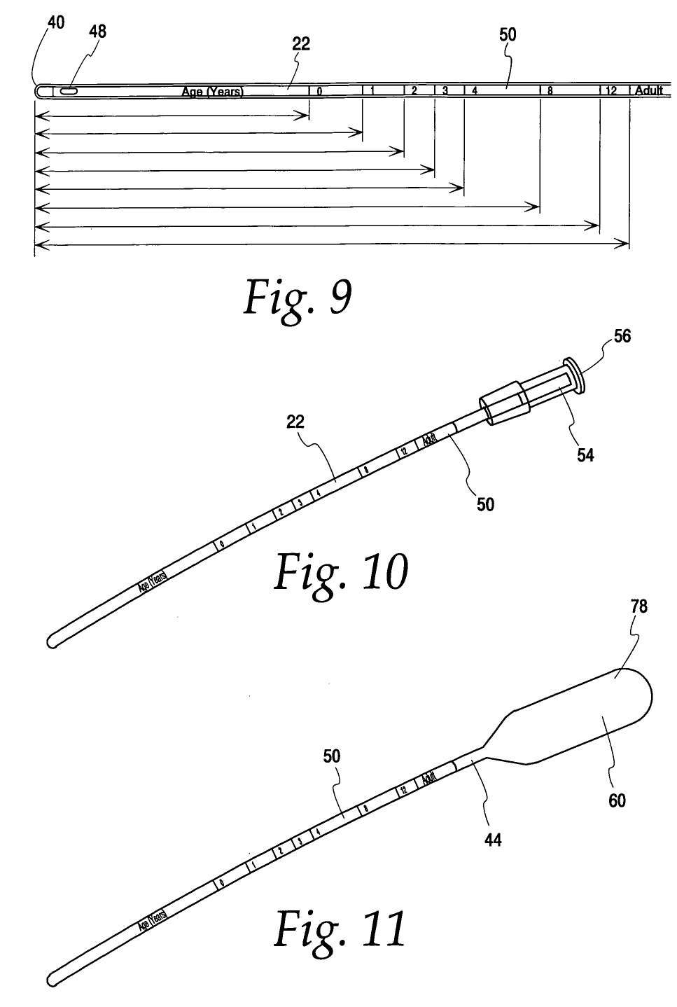

The catheter 22 includes a leading end 40, shaft 42, and connector 44 for connecting to the manually operable suction device 24. The catheter 22 also preferably includes a depth of insertion scale 50 (FIGS. 9, 10, 11) that can be monitored as the catheter 22 is inserted into and guided through the nasal cavity.

Preferably, the catheter leading end 40 is rounded and closed as illustrated in FIGS. 3, 4, 5, 6, and 8. Such a configuration minimizes nasal lining irritation and, importantly, helps enable the catheter 22 to be “self-navigating.” When used properly by a medical professional, the closed and rounded leading end 40 can glance off the patient's middle and lower turbinate and otherwise slide along sensitive nasal tissue toward the back of the nasal cavity without extra manipulation. Such a self-navigating feature results in increased patient comfort and more reliable gathering from the nasopharynx.

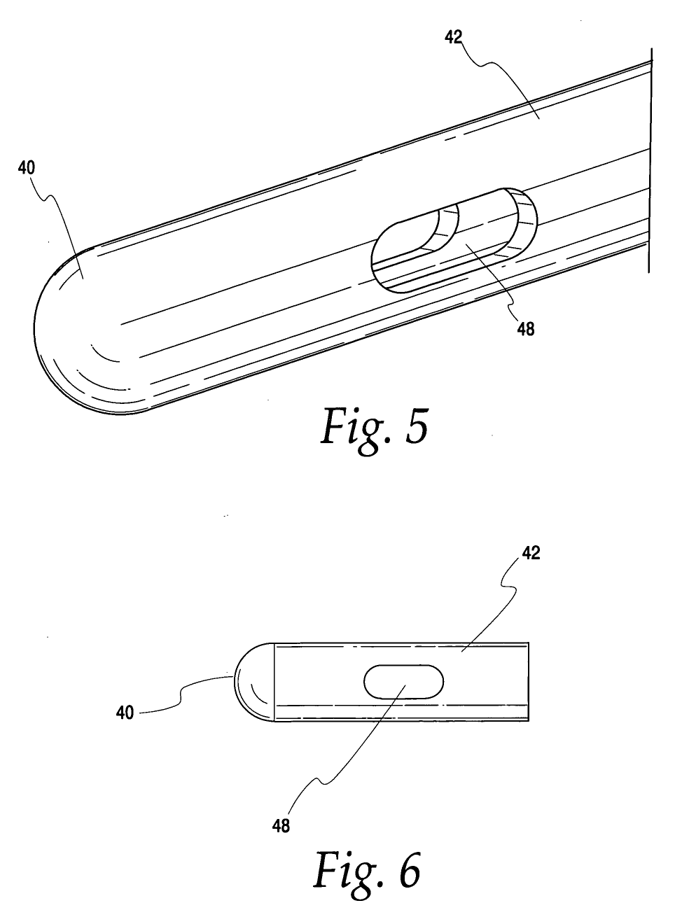

To instill aspiration fluid from the catheter 22, there is at least one aspiration hole 48 defined in the catheter shaft 42 as illustrated in FIGS. 5 and 6. In the illustrated embodiments, there are two such aspiration holes 48 on opposite sides of the shaft 42 from each other. The aspiration holes 48 can be any shape and in any number, but are preferably the oval shape illustrated to allow more aspiration fluid to exit and enter the catheter 22 and quickly obtain nasal fluid specimens. Oval holes also allow larger openings without sacrificing catheter strength during insertion of the catheter 22. The side position of the opening prevents occlusion of the catheter during aspiration compared to an end opening.

In one preferred embodiment illustrated in FIGS. 5 and 6, each aspiration hole 48 is approximately oval in shape, as illustrated, and are approximately 0.110 inches long (plus or minus 0.015 inches) and 0.040 inches wide (plus or minus 0.015 inches).

Also preferably, the aspiration holes 48 are spaced up the catheter shaft 42 from the leading end 40 a distance X (FIG. 6) to prevent weakening of the leading end 40. A preferred spacing X from the extreme end of the leading end 40 is approximately 0.185 inches, but preferably ranges from 0.020 inches to 0.300 inches.

Generally, the aspiration holes 48 must be close enough to the leading end 40 to allow adequate aspiration without a substantial amount of aspirate to be required in the pool 47 or be left behind after the procedure. See: FIG. 13.

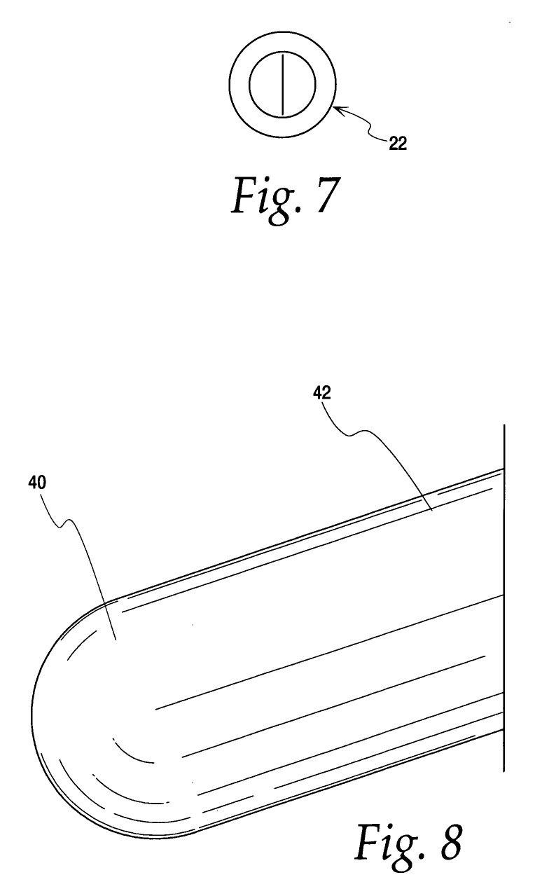

Preferred catheter 22 designs include material durometer (softness) and sidewall thickness to provide sufficient rigidity for reliable insertion while being flexible enough to minimize patient discomfort. In addition, the catheter 22 must not collapse during forceful aspiration. A catheter 22 cross-section is illustrated in FIG. 7. Pebax 3533-SA-01 with a 0.100 inch outside diameter and a 0.065 inch inside diameter is the preferred catheter material and cross-sectional size. Outside diameters up to 0.250 inch and down to 0.030 inch as well as inside diameters from 0.165 inch and down to 0.020 inch could also be used. In addition many durometers could be used from 35-70 Shore D.

Another feature of the present invention is illustrated in FIGS. 9, 10, and 11. The catheter 22 preferably includes an anatomic nasopharyngeal depth calibration scale 50. Using this scale 50, a health care provider can monitor the catheter's insertion depth before beginning aspiration. Studies of using Cat and MRI scans have been performed on patients ranging from newborns to adults.

Average distances from nasal openings to the back (posterior) nasopharyngeal wall were measured and plotted to derive the preferred insertion depth scale by age illustrated in FIG. 9. Such a scale is preferably pad printed on the catheter shaft 42 to provide a reliable insertion depth measurement without guesswork or external measuring devices. The scale 50 ensures proper insertion depth for the catheter 22 without unnecessary patient discomfort. In particular, such a scale spares the patient and health care provider from using patient discomfort levels or guesswork as a guide to insertion depth.

In addition, the scale 50 reduces the chance that the catheter 22 will be inserted beyond nasopharynx to the oropharynx. Over-insertion of the catheter 22 could result in aspiration of saline, coughing, and choking during the procedure.

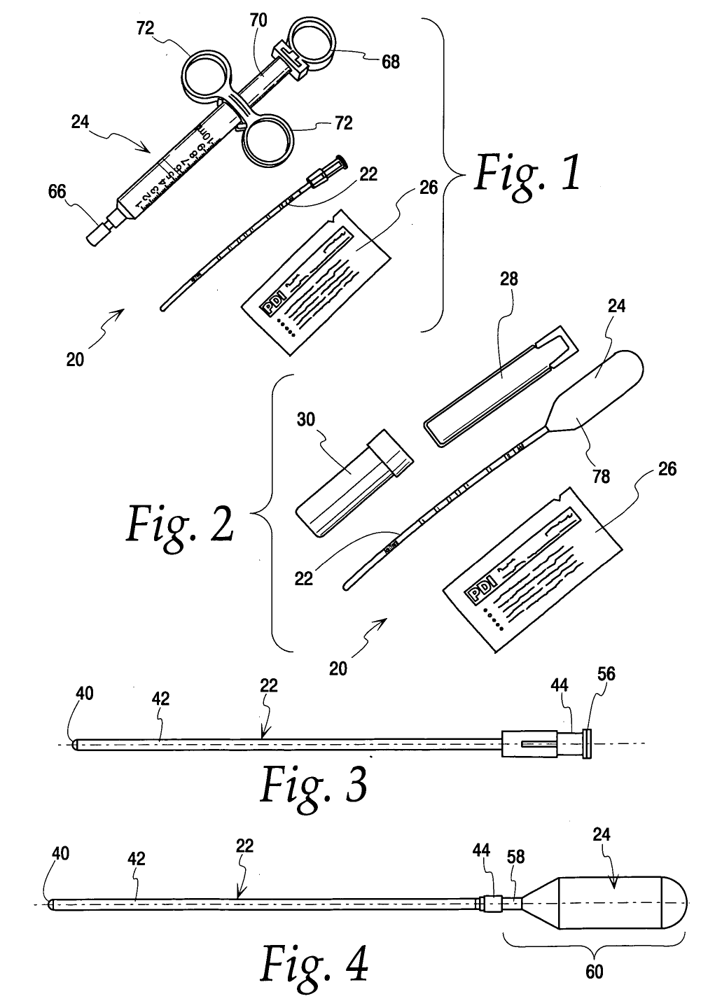

Referring back to FIGS. 3 through 4, there is illustrated two possible catheter connector 44 designs although other connectors not illustrated could be used in the present invention. The connectors 44 are used to quickly and reliably connect the catheters 22 to the manually operably suction devices 24. In FIGS. 1, 3 and 10, there is illustrated a Luer catheter 54, having a “Luer lock” 56. Such a lock 56 is a standard in the medical industry, so medical personnel are familiar with its operation and confident in its reliability.

Another catheter connector 44 is illustrated in FIG. 4 as being a simple friction fit over a nipple 58 that uses the resiliency of the catheter 22 to connect to a manually operable suction device 24 such as the pipette 60 illustrated. This connection is simple and reliable.

Manually operable suction devices 24 are illustrated in FIGS. 1 and 2. In FIG. 1 the suction device 24 is a syringe 64, and is preferably a control syringe having a connector 66 to mate with a luer lock 56, as described above in relation to FIG. 3.

A control syringe 64 as illustrated in FIGS. 1 and 13 includes a thumb hole 68 on the plunger 70 and finger holes 72 on the syringe 64 to provide improved control over the procedure using one hand quickly and comfortably.

Another embodiment of a manually operable suction device 24 in accordance with the present invention illustrated in FIGS. 2 and 11 includes a bulb aspirator 78, which are smaller and more compact than syringes. Bulb aspirators 78 such as pipettes or other flexible bulbs provide adequate control of the procedure, and yet has less aspiration force compared to a syringe. The bulb aspirator 78 can be used as an aspirant or as the saline container. When the pipette or bulb aspirator 78 is used it may be pre-filled with aspirant (such as saline solution.) When such a design is used it could be provided with a twist off, cuttable or pull off section just outside of a lure lock fitting which is molded onto the pipette 78. This provides an even more simple system of a lure catheter, pipette, lubricant, and a cap.

Other manually operable suction devices can be used with the present invention.

The kit 20 may also include lubricant 26 for the catheter 22 and may include aspiration fluid 28 such as saline solution. Also, the kit 20 preferably includes a specimen container 30 that is sized to hold the nasal fluid specimen. When either a syringe 64 or pipette 78 is used as the manually operable suction device 24, the syringe 64 or bulb aspirator 78 itself can be used as a specimen container, and no additional containers need be provided. Optionally, more than one container 30 can be provided when multiple tests may be performed on the sample.

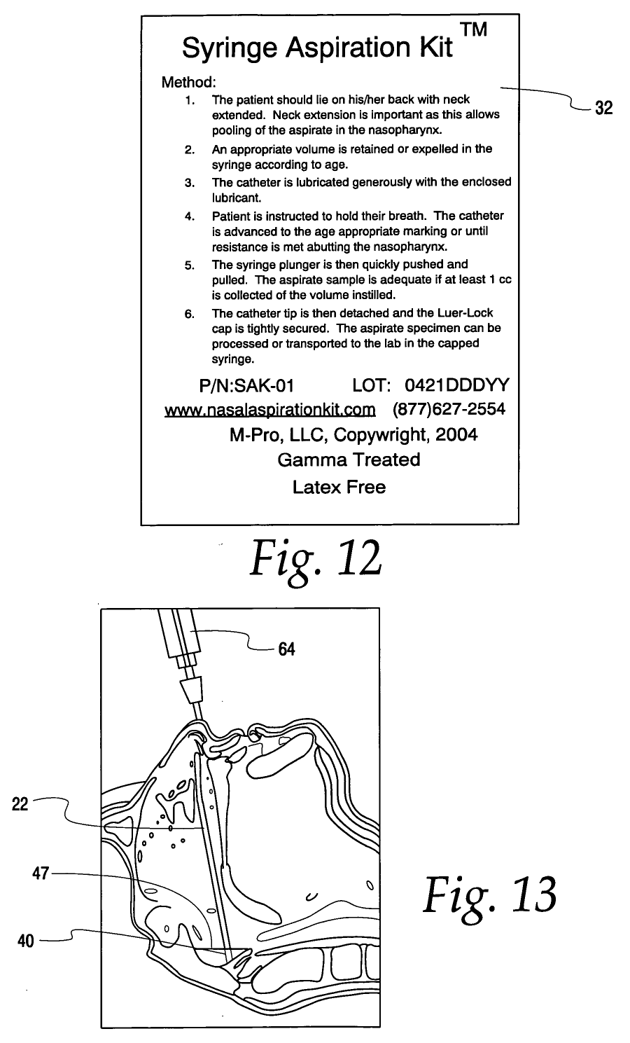

The kit 20 also preferably includes an instruction label 32 or insert as illustrated in FIG. 12. The instructions 32 describe the necessary steps as: having a patient lie on his or her back (supine position); extending the patient's neck to allow pooling of the aspirate in the nasopharynx; drawing into the catheter 22 sufficient aspiration fluid 28, lubricating the catheter; instructing the patient to hold his or her breath; advancing the catheter until the appropriate insertion depth is noted on the depth insertion scale 50 or until resistance is met by the nasopharynx, quickly pushing the plunger 70 (or squeezing the aspiration bulb 78) and then pulling the plungers 70 (releasing pressure from the aspiration bulb 78) to withdraw a specimen (preferably about 1 cc); disconnecting the catheter 22 from the manually operable suction device 24 to retain the specimen in the suction device 24 or otherwise depositing the specimen in the specimen container 30 for transport to an appropriate lab for testing.

A kit in accordance with the present invention is available from M-Pro, LLC located at 5255 East River Road, Suite 210, Fridley, Minn. 55421, phone (763) 258-8170.

Modifications to the foregoing detailed description of the preferred embodiments will be apparent to those skilled in the art and no unnecessary limitations there from should be read into the following claims.

1. A nasal aspiration kit for obtaining a nasopharyngeal sample, the kit comprising:
a manually operable suction device; and
a catheter for connection to the manually operable suction device for navigating to a patient's nasopharynx.

2. The kit of claim 1, wherein the manually operable suction device is a syringe.

3. The kit of claim 1, wherein the manually operable suction device is a control syringe.

4. The kit of claim 1, wherein the manually operable suction device is a bulb aspirator.

5. The kit of claim 1, wherein the catheter comprises:
a Luer catheter.

6. The kit of claim 1, wherein the catheter comprises a friction-fit connector for mating with the manually operable suction device.

7. The kit of claim 1, wherein the catheter has an external diameter in the range of about 0.100 inches.

8. The kit of claim 1, wherein the catheter defines an internal diameter in the range of about 0.065 inches.

9. The kit of claim 1, wherein the catheter has a durometer in the range of about 35-70 Shore A.

10. The kit of claim 1, wherein the catheter comprises:
a lead end; and
the lead end is substantially self-navigating through the nasal cavity.

11. The kit of claim 1, wherein the catheter comprises:
a lead end; and
a shaft, the shaft defining an aspiration hole spaced apart from the lead end and through which aspirant and nasal samples can flow.

12. The kit of claim 1, wherein the catheter comprises:
a lead end, the lead end is rounded and closed; and
a shaft defining an aspiration hole.

13. The kit of claim 12, wherein the hole is about 0.110 inches long and 0.040 inches wide.

14. The kit of claim 12, wherein the hole is about 0.185 inches from the lead end.

15. The kit of claim 1, wherein the catheter comprises:
an insertion scale for determining insertion depth of the catheter.

16. The kit of claim 1, wherein the catheter comprises:
an age calibration scale for approximately an appropriate catheter insertion depth based on patient age.

17. The kit of claim 1, wherein the catheter comprises:
a closed and rounded lead end;
a shaft defining an aspiration hole spaced apart from the closed and rounded lead end; and
an insertion scale for determining insertion depth of the catheter.

18. The kit of claim 1, and further comprising:
a specimen transport container.

19. The kit of claim 1, and further comprising:
a specimen transport container;
a lubricant for application to the catheter prior to insertion; and
an aspirant.

20. The kit of claim 1, and further comprising:
use instructions, wherein the use instructions comprise the steps of:
instructing a patient to lie on his/her back with neck extended;
lubricating the catheter;
inserting aspirant into the manually operable suction device;
advancing the catheter into the patient's nasal cavity;
injecting aspirant into the nasal cavity;
withdrawing a nasopharyngeal specimen into the catheter; and
retaining the specimen for testing.

21. The kit of claim 20, wherein the use instructions further comprise instructions to have the patient hold his/her breath prior to advancing the catheter into the patient's nose and nasal cavity.

22. A method for obtaining a nasopharyngeal fluid sample from a patient, the method comprising the steps of:
inserting a catheter into the nasal cavity of a patient;
instilling an aspirant into the patient's nasal cavity; and
withdrawing a specimen from the patient's nasopharynx using a manually operable suction source.

23. The method of claim 22, wherein the step of inserting a catheter into the patient's nasal cavity comprises the step of:
inserting a self-navigating catheter into the patient's nasal pharnyx.

24. The method of claim 22, wherein the catheter being inserted comprises:
a closed leading end.

25. The method of claim 22, wherein the catheter being inserted comprises:
a closed leading end and defines an aspiration hole spaced apart from the leading end.

26. The method of claim 22, wherein the catheter being inserted comprises:
a closed leading end, and defines a pair of aspiration holes spaced part from the leading end.

27. The method of claim 22, wherein the catheter being inserted has an outside diameter of about 0.100 inches.

28. The method of claim 22, wherein the catheter being inserted defines an internal diameter of about 0.065 inches.

29. The method of claim 22, wherein the catheter being inserted has a closed leading end and defines an aspiration hole spaced about 0.185 inches apart from the closed leading end.

30. The method of claim 22, wherein the catheter being inserted has a closed leading end, and defines an aspiration hole about 0.110 inches long and about 0.040 inches wide.

31. The method of claim 22, wherein the catheter being inserted comprises a depth of insertion scale.

32. The method of claim 22, wherein the catheter being inserted comprises:
an age calibration scale for approximating an appropriate insertion depth for catheter based on patient age.

33. The method of claim 22, wherein the catheter being inserted comprises:
a closed leading end;
an aspiration hole spaced apart from the closed leading end; and
a depth of insertion scale.

34. The method of claim 22, wherein the catheter being inserted comprises:
a closed leading end;
an aspiration hole spaced apart from the closed leading end; and
an age calibration scale for approximating an appropriate depth of catheter insertion based on patient age.

35. The method of claim 22, wherein the manually operable suction source is a syringe.

36. The method of claim 22, wherein the manually operable suction source comprises a control syringe.

37. The method of claim 22, wherein the manually operable suction source comprises:
a bulb aspirator.

38. The method of claim 22, wherein the catheter and manually operable suction source are removably joined together.

39. The method of claim 22, wherein the catheter and manually operable suction source are formed integrally with one another.

40. The method of claim 22, and further comprising the step of:
lubricating the catheter prior to insertion into the nasal cavity.

41. The method of claim 22, and further comprising the step of:
storing a sample of nasopharyngeal fluid in a specimen container.
